(19) United States
(12) Patent Application Publication
(10) Pub. No.: US 2025/0049880 A1
(43) Pub. Date: Feb. 13, 2025

(54) COMPOSITIONS FOR ENHANCING OXYGEN PRODUCTION AND RELATED METHODS

(71) Applicant: MARCON FORMULATIONS LLC, Quantico, VA (US)

(72) Inventor: Marcus SMALLS, McClellanville, SC (US)

(21) Appl. No.: 18/231,978

(22) Filed: Aug. 9, 2023

(51) Int. Cl. A61K 36/87; A61K 36/41; A61K 36/67; A61K 36/68; A61K 36/71; A61K 36/81; A61K 36/9068

(52) U.S. Cl. CPC A61K 36/87; A61K 36/41; A61K 36/67; A61K 36/68; A61K 36/71; A61K 36/81; A61K 36/9068

(57) ABSTRACT

Nutritional compositions and methods are provided that minimize the effects of hypoxia, fatigue, stress and anxiety, enhance endurance and blood flow while promoting mental focus in an individual using a minimal number of active ingredients at or near threshold active dosages, wherein such compositions comprise grape seed extract and Withania somnifera, and optionally further include Rhodiola rosa, Nigela sativa, Zingiber officinale, Bacopa monnieri, and black pepper.

### FIELD OF THE INVENTION

The present invention relates to a novel nutritional composition which provides for enhancing oxygen production while minimizing the effects of hypoxia, minimizing fatigue, stress and anxiety, enhancing endurance and blood flow, promoting mental focus in an individual and a method for achieving same by consuming the nutritional composition.

### BACKGROUND

Hypoxia is a condition in which the body is deprived of an adequate oxygen supply needed to sustain cognitive brain function and prevent tissue and organ damage. Hypoxia has been known to occur in healthy people during flight because the percentage of oxygen in inspired air is constant at different altitudes, but the fall in atmospheric pressure at higher altitude decreases the partial pressure of inspired oxygen and hence the driving pressure for gas exchange in the lungs. Hypoxia can lead to high altitude pulmonary edema (HAPE), high altitude cerebral edema (HACE), and hypoxic loss of consciousness (HLOC).

Cabin pressurization and onboard oxygen systems have allowed for high altitude aviation activities by aircrew, within the aircraft. However, flight safety is still a concern with regard to hypoxia because of the potential for failure of the onboard flight oxygen equipment and failure of the integrity of the aircraft cabin itself. Commercial aircraft maintain a cabin pressure of about 11 to 12 pounds per square inch simulating the pressure equivalent to between 6,000 to 8,000 ft. in elevation. Unpressurized aircraft are required to operate below a 10,000 ft. altitude ceiling to prevent hypoxia from occurring. Combat aircraft have a higher altitude ceiling due to their onboard oxygen systems. Nevertheless, the threat of hypoxia is a risk in the event of an accidental loss of cabin pressurization, or when flying unpressurized aircraft beyond an altitude in which the ambient air does not have sufficient oxygen for breathing.

Aviation related hypoxia is known as hypobaric hypoxia, which is caused by breathing air at altitudes above 10,000 feet. The partial pressure of oxygen in the inspired air is progressively reduced when altitude increases. Thus, accidental hypoxia can occur when ascending to a high altitude without a supplemental oxygen supply, or the failure of personal or aircraft oxygen breathing equipment, or loss of aircraft cabin pressure.

The signs and symptoms of hypoxia become apparent as the degree of hypoxia increases. For example, the symptoms include shortness of breath, air hunger, excessive yawning, tiredness, fatigue, euphoria, physical impairment, or mental impairment, which can ultimately lead to a complete loss of consciousness.

In order to minimize the effects of hypoxia, methods include acclimatizing slowly through a transition of progressively higher altitudes. The body will attempt to adjust to higher altitudes having the lower oxygen levels by increasing respiratory volume, increasing pulmonary artery pressure, cardiac output, the number of red blood cells, oxygen carrying capability of red blood cells, and by even changing body tissues to promote normal function at lower oxygen levels. However, acclimation is not always successful or even possible in emergency situations.

The current invention aims to enhance oxygen production while minimizing many of the symptoms associated with hypoxia and provide a composition and method to minimize fatigue, stress and anxiety, enhance endurance and blood flow while promoting mental focus in an individual.

### SUMMARY

The present invention, in accordance with one embodiment thereof, provides for a novel nutritional composition for enhancing oxygen production while minimizing the effects of hypoxia, fatigue, stress and anxiety, enhancing endurance and blood flow, promoting mental focus in an individual e.g., a human or animal. The present invention, in accordance with one embodiment thereof, also provides, by the ingestion of the novel nutritional composition, a method for enhancing oxygen production while minimizing the effects of hypoxia, fatigue, stress and anxiety, enhancing endurance and blood flow, promoting mental focus in an individual.

In one aspect of the inventive subject matter, a nutritional supplement for enhancing oxygen production while minimizing the effects of hypoxia, fatigue, stress and anxiety, enhancing endurance and blood flow, promoting mental focus includes a therapeutically effective dosage of grape seed extract (95% polyphenols) preferably 50-500 mg, and Withania somnifera (5% withanolides) preferably 125-1000 mg.

Contemplated supplements may still further include one or more additional ingredients comprising (i) Rhodiola rosa (3% rosavins and 1% salidroside) preferably in an amount 125-1000 mg, (ii) nigella sativa (5% thymoquinone) preferably in an amount 200-1000 mg, (iii) Zingiber officinale (20% gingerols and shogoals) preferably in an amount of 100-1500 mg, (iV) Bacopa monnieri (50% bacosides) preferable in an amount 100-750 mg, and (v) black pepper (95% piperine) preferably in an amount 5-15 mg.

Consequently, methods of assisting enhancement of oxygen production and blood flow while promoting mental focus to minimize the effects hypoxia in a person using the nutritional supplement discussed above are also contemplated. The present invention, in accordance with one embodiment thereof, also provides, by the ingestion of the novel nutritional composition, a method for enhancing oxygen production while minimizing the effects of hypoxia, fatigue, stress and anxiety, while enhancing endurance and blood flow and promoting mental focus of an individual. Inactive ingredients may be included and typically comprise a carrier, a binder, an excipient, and/or a dye. Moreover, the nutritional supplement may be associated with information stating that the nutritional supplement enhances blood flow while minimizing the effects of hypoxia and enhances blood flow and endurance and minimizes fatigue, stress and anxiety, and where desired. Additionally, it is generally preferred that the dosage unit of the supplement is equal to or less than 1,600 mg.

Various objects, features, aspects and advantages of the present invention will become more apparent from the following detailed description of preferred embodiments of the invention.

### DETAILED DESCRIPTION

The invention provides for a nutritional composition including grape seed extract, Withania somnifera, Rhodiola rosea, nigella sativa, Zingiber officinale, Bacopa monnieri and black pepper. The present invention, in accordance with one embodiment thereof, also provides, by the ingestion of the novel nutritional composition, a method for enhancing oxygen production while minimizing the effects of hypoxia, fatigue, stress and anxiety, enhancing endurance and blood flow, promoting mental focus in an individual.

As used herein, “an effective amount” refers to an amount effective for enhancing oxygen production while minimizing the effects of hypoxia, fatigue, stress and anxiety, enhancing endurance and blood flow, while promoting mental focus in an individual when administered over a period of weeks in accordance with a predetermined regimen set forth in greater detail below. An effective amount of the nutritional composition is preferably equal or less than 1,200 mg, more preferably equal or less than 1,600 mg, and even more preferably equal or less 2,000 mg, and most preferably equal or less than 2,400 mg.

The invention provides for a nutritional composition including grape seed extract, Withania somnifera, Rhodiola rosea, nigella sativa, Zingiber officinale, Bacopa monnieri and black pepper. The present invention, in accordance with one embodiment thereof, also provides, by the ingestion of the novel nutritional composition, a method for enhancing oxygen production while minimizing the effects of hypoxia, fatigue, stress and anxiety, enhancing endurance and blood flow, promoting mental focus in an individual.

### Grape Seed Extract

Hypoxia induces a wide range of deleterious effects at the cellular level due to an increased production of reactive oxygen species (ROS). Polyphenols from grape seeds, which are potent antioxidants might protect the brain against oxidative stress produced by hypobaric hypoxia.

In order to obtain grape seed extract (GSE), grape seeds are separated from the grapes, air-dried and grounded to fine powder. The grounded grape seed powder is macerated in 70% ethanol and filtered. The filtrate is dried to evaporate ethanol to obtain powdered GSE. Dried seeds of grapes contain around 35% fiber along with 29% extractable components including Phenolic compounds, proteins (11%), mineral (3%) and water (7%). GSE has an abundant source of polyphenols. Polyphenols and flavonoids present in the GSE have been shown remarkable interest based on positive reports of their antioxidant properties and ability to serve as free radical scavengers. Grape seed polyphenols have a higher antioxidant activity as compared to other well-known antioxidants (such as vitamin C, vitamin E, and b-carotene). Besides their antioxidant activity, it also contains some enzymes that catalyze the release of histamine during inflammation and allergies. The amount of oil present in grape seed depends on the variety of the grape (usual range 10-16% of dry weight).

Grape seed oil also contains a high amount fatty acids (unsaturated) ranging from 85 to 90% such as a-linolenic acid (x-3) and c-linolenic acid (x-6). These fatty acids are related to a reduction of cardiovascular disease, cancer, hypertension, and autoimmune disorders. An oil containing high amounts of linoleic acid results in the reduction of total blood cholesterol and low-density lipoprotein (LDL) cholesterol. LDL cholesterol is responsible for the formation of arteriosclerosis; therefore, the application of grape seed oil creates a positive effect in the reduction of arteriosclerosis.

Fatty acid compositions of GSE include linoleic acid, linolenic acid, oleic acid and palmitic acid, and are very important in lipid metabolism. The major fatty acid in GSE is linoleic acid followed by oleic acid and palmitic acid. The seeds and peels of grapes also contain considerable portions of dietary fiber that lowers the risks of colon cancer, heart disease, diabetes and obesity.

Withania somnifera

Withania somnifera is an evergreen woody shrub, commonly known as Ashwagandha. It has been used extensively as an herbal drug in the Ayurvedic and Unani systems of medicine for the last 3,000 years. It grows abundantly in arid areas stretching from the Mediterranean across tropical Africa, South Africa, and the Canary and Cape Verde Islands, as well as Afghanistan, Baluchistan, Pakistan, Sri Lanka, China, Nepal, and India. It is grown in gardens in warmer parts of Europe and has emerged as a natural weed in South Australia and New South Wales.

In India, it is mostly grown for its fleshy roots, which contain a profusion of phytoconstituents with a multitude of therapeutic values. The plant is widespread in India's arid areas, notably in Punjab, Gujarat, Uttar Pradesh, Maharashtra, West Bengal, and Rajasthan. It has been used in traditional system of medicine as an anti-stress, narcotic, diuretic, combating anemia, aphrodisiac, etc., for constipation, against worms, liver disease, leprosy, anti-inflammatory, cardiovascular problems, joint pain, antibacterial, nervous system disorders, arthritis, etc.

Various pharmacological activities have been reported in Withania somnifera, including anti-inflammatory, analgesic, anti-arthritic, hepatoprotective, anti-cancer, anti-epileptic, cardioprotective, neuroprotective, anti-microbial, anti-fungal, antioxidant, immunomodulatory, anti-depressant, anti-diabetic, anti-platelet, fibrinolytic, etc. The major bioactive compounds reported in Withania somnifera are the steroidal lactones called withanolides, the most important ones that are responsible for various bioefficacies are withaferin-A, withanolide-D, and withanone. These bioactive compounds are known to impart pharmacological effects by targeting different biomolecules in the living systems.

They are potent antioxidants that quench free radicals and other reactive oxygen species (ROS) and inhibit free radical induced cell damage. These control the expression of various enzymes, receptors, and other regulatory proteins which are involved in the pathogenesis of various diseases by upregulation and downregulation of transcription factors which controls the production of these regulatory macromolecules.

### Rhodiola Rosea

Extract from the roots and rhizomes of Rhodiola rosea is an adaptogen that acts to increase the body's resistance to stress, exhaustion and fatigue. It is the main adaptogen given the indication stress. A wide variety of preclinical in vivo and ex vivo studies conducted in cell lines and animal models have elucidated the presence of several biochemical and pharmacological stress-reducing actions of Rhodiola rosea. It possesses a unique ability to normalize the release of stress hormones while simultaneously boosting energy metabolism via activation of ATP synthesis in mitochondria.

Rhodiola rosea acts to normalize cortisol synthesis potentially through inhibition of stress activated protein kinase (SAPK) pathway involved in the pathogenesis of glucocorticoid resistance, which is also found in certain chronic immune/inflammatory diseases. At the same time, through inhibition of the SAPK pathway, Rhodiola rosea potentially prevents the formation of nitric oxide (NO) and the associated decline in adenosine triphosphate (ATP) synthesis.

Stress leads to a damage of the mitochondrial function and the excessive production of reactive oxidative species (ROS) in mitochondria, which may cause damage e.g., to proteins, nucleic acids, and membranes, which in turn can lead to activation of cell death processes such as apoptosis. Rhodiola rosea may also offer potential protection against heart and brain diseases (e.g., heart attack, stroke, depression and Alzheimer's disease) through anti-oxidative/anti-inflammatory mechanisms.

### Nigella Sativa

Nigella sativa, also called black cumin or black seeds, is famous for its culinary uses and is historically precious in traditional medicine. Black cumin is native to a vast region of the eastern Mediterranean, northern Africa, the Indian subcontinent, and Southwest Asia, and is cultivated in many countries, including Egypt, Iran, Greece, Syria, Albania, Turkey, Saudi Arabia, India, and Pakistan. Being a panacea, black cumin, in the form of essential oil, paste, powder, and extract, has been indicated in traditional medicine for many diseases/conditions, such as asthma, bronchitis, rheumatism, headache, back pain, anorexia, amenorrhea, paralysis, inflammation, mental debility, eczema, and hypertension, to name a few.

These traditional uses of Nigella sativa seeds are largely attributed to their wide array of medicinal properties, including antioxidant, anti-inflammatory, immunomodulatory, anticancer, neuroprotective, antimicrobial, antihypertensive, cardioprotective, antidiabetic, gastroprotective, nephroprotective and hepatoprotective properties. Black cumin seed, particularly its essential oil, contains thymoquinone (TQ), thymohydroquinone, thymol, carvacrol, nigellidine, nigellicine, and a-hederin, which are mostly responsible for its pharmacological effects and therapeutic benefits.

### Zingiber Officinale

Zingiber officinale is the most common ginger species, contains 80-90 nonvolatile compounds that have anti-inflammatory, antioxidant, and antiemetic effects, as well as lowering blood pressure, blood lipid, and blood glucose. The myriad of mechanisms of action have been extensively examined in animal and cell models, mostly involving gingerol, shogaol, zingerone, gingerdiol, and paradol compounds.

Briefly, the anti-inflammatory effects of ginger have been linked to reducing pain and the vasodilatory effects to lowering blood pressure. Ginger has been found to inhibit the production of cholesterol as well as adipocytes, thus benefiting the blood lipid profile and weight management, respectively. Ginger compounds have also been shown to act similarly to hypoglycemic agents in assisting with transportation of glucose into cells, as well as antiemetic medications to block the activation of receptors that initiate nausea and vomiting pathways.

### Bacopa Monnieri

Bacopa monnieri (Brahmi) is a well-known perennial, creeping herb possessing bioactive formulation in the Indian Ayurveda system, implicated in the therapeutic management of numerous diseases. This herb was used by Ancient Vedic scholars due to its pharmacological effect, especially as a nerve tonic and nootropic booster. Bioactive components of Brahmi belong to alkaloids, saponins, flavonoids, triterpenes and cucurbitacin, having potential role in neuroprotection.

The extracts of Bacopa monnieri are well-recognized for their antioxidant activity with numerous modes of action to protect the brain against oxidative damage and cognitive decline in the elderly. The cognition-promoting roles of Bacopa monnieri can be due to the antioxidant effects of alcoholic extracts and bacoside. The extract of Bacopa monnieri and its isolated valuable therapeutic agents have been extensively investigated for their nootropic effects, antioxidant, antimicrobial properties and analgesic activity, etc. These traditional pharmacological claims have been bolstered by large-scale research and clinical studies. Bacopa monnieri has been the focus of research as a versatile therapeutic agent for various disorders and neurodegenerative diseases.

### Black Pepper

Black pepper or Piper nigrum is associated with black peppercorns and berries used for seasoning of different dishes. In general, black pepper mainly contains various alkaloids, volatile oils, carbohydrates, starch, and proteins. Black pepper is known to be a source of an important alkaloid piperine, which adds a strong, pungent flavor to dishes. However, black pepper has also been known for many centuries to treat different types of health problems, including intermittent fever, influenza, muscular pain, and migraines because it contains numerous active ingredients like flavonoids, terpenoids, phytoestrogens and minerals.

Piperine from black pepper has been demonstrated during in vitro experiments to protect against oxidative damage by inhibiting or quenching free radicals and reactive oxygen species and inhibit lipid peroxidation. For example, Piperine was found to act as a hydroxyl radical scavenger at low concentrations. Piperine has a low risk of toxicity, is not genotoxic and does not present any significant adverse effects on internal organs, weight, Hb levels, total serum proteins, albumin, cholesterol, fats, and nitrogen. Its various medical properties and advantageous safety profiles facilitate the pharmaceutical employment of piperine as a dietary and health supplement.

### Inventive Composition

A nutritional composition is prepared that includes an effective amount of grape seed extract (95% polyphenols) preferably 50-500 mg, and Withania somnifera (5% withanolides) preferably 125-1000 mg. Additionally, such formulations may further include Rhodiola rosea (3% rosavins and 1% salidroside) preferably in an amount 125-1000 mg, nigella sativa (5% thymoquinone) preferably in an amount 200-1000 mg, Zingiber officinale (20% gingerols and shogoals) preferably in an amount of 100-1500 mg, Bacopa monnieri (50% bacosides) preferably in an amount 100-750 mg, black pepper (95% piperine) preferably in an amount 5-15 mg. The following table illustrates exemplary compositions.

| | Min | Max |
|---|---|---|
| Grape Seed Extract (GSE) | 50 mg | 500 mg |
| Withania somnifera | 125 mg | 1000 mg |
| (Ashwagandha) | | |
| (Additional Ingredients) | — | — |
| (i) Rhodiola rosea | 125 mg | 1000 mg |
| (ii) Nigella sativa | 200 mg | 1000 mg |
| (iii) Zingiber officinale | 100 mg | 1500 mg |
| (iv) Bacopa monnieri | 100 mg | 750 mg |
| (v) Black pepper | 5 mg | 15 mg |

Most preferably, the effective daily dosage is administered between once daily and four times daily in dosage units of accordingly adjusted weight. While not limiting to the inventive subject matter, grape seed extract and Withania somifera in contemplated formulations is thought to act as an antioxidant.

Therefore, and viewed from a different perspective, contemplated nutritional supplements for enhancing blood flow include (a) grape seed extract between 50-500 mg, and (b) Withania somifera between 125-1000 mg.

Contemplated supplements may still further include one or more additional ingredients comprising (i) Rhodiola rosa (3% rosavins and 1% salidroside) preferably in an amount 125-1000 mg, (ii) Nigela sativa (5% thymoquinone) preferably in an amount 200-1000 mg, (iii) Zingiber officinale (20% gingerols and shogoals) preferably in an amount of 100-1500 mg, (iV) Bacopa monnieri (50% bacosides) preferable in an amount 100-750 mg, and (v) black pepper (95% piperine) preferably in an amount 5-15 mg.

Furthermore, contemplated supplements may include inactive ingredients, which may help in formulation, disintegration, or other manner. Therefore, suitable inactive ingredients include carriers, binders, excipients, dyes, etc. Oral formulation is typically in form of a liquid or powder, or gel, or a solid form, and most preferably in form of tablet, pill, capsule, or softgel which may or may not have an enteric coating, such coating allowing for the ingredients to by-pass the upper GI tract where gastro-intestinal disturbances can be problematic for some individuals. Moreover, one or more of the active ingredients may be in slow release formulation to extend release over a period of between 1-24 hours. In less preferred aspects, the supplement may also be formulated as a liquid or a gel, or embedded in a dissolvable film or chewing preparation.

It is further preferred that the supplement is formulated such that the daily dosage unit of the supplement is equal or less than 1,200 mg, more preferably equal or less than 1,600 mg, and even more preferably equal or less 2,000 mg, and most preferably equal or less than 2,400 mg, wherein administration may be between once daily and ten times daily. Therefore, suitable oral single dosage forms may preferably have a weight between 200 mg and 600 mg.

Regardless of the actual weight of the single dosage form, it is preferred that the grape seed extract (GSE) and Withania somnifera together account for at least 80 wt %, more preferably at least 85 wt %, of a dosage unit of the supplement.

Consequently, a method of enhancing oxygen production while minimizing the effects of hypoxia in a person using a nutritional supplement includes a step of providing contemplated compositions for oral administration under a schedule and protocol effective to enhance blood flow and mental focus.

Many modifications and other embodiments of the invention will come to the mind of one skilled in the art having the benefit of the teachings presented in the foregoing descriptions. Therefore, it is understood that the invention is not to be limited to the specific embodiments disclosed, and that modifications and embodiments are intended to be included within the scope of the appended claims.

That which is claimed is:

1. A nutritional composition for enhancing oxygen production while minimizing the effects of hypoxia, minimizing fatigue, stress and anxiety, enhancing endurance and blood flow, and promoting mental focus in an individual, the composition comprising an effective amount of grape seed extract and Withania somnifera.

2. The nutritional composition of claim 1, further comprising Rhodiola rosea.

3. The nutritional composition of claim 1, further comprising nigella sativa.

4. The nutritional composition of claim 1, further comprising Zingiber officinale.

5. The nutritional composition of claim 1, further comprising Bacopa monnieri.

6. The nutritional composition of claim 1, further comprising black pepper.

7. The nutritional composition of claim 1, wherein the composition is provided in the form of capsules, pills, tablets, soft gel or powder.

8. A method for enhancing oxygen production while minimizing the effects of hypoxia, minimizing fatigue, stress and anxiety, enhancing and blood flow, promoting mental focus in an individual by administering a nutritional composition comprising an effective amount of grape seed extract and Withania somnifera.

9. The method of claim 8, further comprising Rhodiola rosea.

10. The method of claim 8, further comprising nigella sativa.

11. The method of claim 8, further comprising Zingiber officinale.

12. The method of claim 8, further comprising Bacopa monnieri.

13. The method of claim 8, further comprising black pepper.

14. The method of claim 8, wherein the nutritional composition is provided in the form of capsules pills, tablets, caplets, soft gel or powder.
